(19) United States
(12) Patent Application Publication
(10) Pub. No.: US 2011/0112614 A1
(43) Pub. Date: May 12, 2011

(54) FIBER REINFORCED SILICONE FOR CARDIAC AND NEUROSTIMULATION LEADS

(76) Inventor: Joshua Haarer, Hugo, MN (US)

(21) Appl. No.: 12/886,995

(22) Filed: Sep. 21, 2010

Related U.S. Application Data
(60) Provisional application No. 61/260,631, filed on Nov. 12, 2009.

(51) Int. Cl. A61N 1/00

(52) U.S. Cl. 607/116

(57) ABSTRACT

A least a portion of a lead body includes a fiber reinforced silicone elastomer. The fiber reinforced silicone elastomer comprises a uniform dispersion of discrete fibers having a random orientation. The fiber reinforced silicone elastomer may demonstrate an improvement in mechanical properties such as, for example, stiffness and strength, when compared to an analogous non-reinforced silicone elastomer. As a result, lead bodies having reduced outer diameters may be fabricated without a decrease in the overall mechanical strength and stiffness of the material.

### CROSS REFERENCE TO RELATED APPLICATIONS

This application claims the benefit under 35 U.S.C. §119 of U.S. Provisional Application No. 61/260,631, filed on Nov. 12, 2009, entitled “Fiber Reinforced Silicone for Cardiac and Neurostimulation Leads,” which is incorporated herein by reference in its entirety for all purposes.

### TECHNICAL FIELD

The present invention relates to medical electrical leads. More particularly, the present invention is related to a medical electrical lead body incorporating a fiber reinforced silicone elastomer.

### BACKGROUND

Implantable medical devices for treating irregular contractions of the heart with electrical stimuli are well known as are implantable medical devices for modulating the peripheral or central nervous systems. Exemplary implantable devices are defibrillators, pacemakers and neurostimulators. Various types of electrical leads for these devices have been suggested. Such leads have an elongated, flexible body and are introduced into the patient's body through various means to access target sites for therapy. Therapy can be delivered to either the right side or the left side of the heart and to the peripheral or central nervous system.

Recently, there has been an effort to reduce the outer diameter of medical electrical lead bodies including endocardial, epicardial, defibrillation, and neurostimulation leads. Reduction in lead body diameter can facilitate placement of the lead at a target location within a patient's body.

### SUMMARY

Discussed herein are various components for implantable medical electrical leads including a fiber-reinforced silicone elastomer comprising discrete fibers having a random orientation uniformly dispersed within a silicone elastomer matrix, as well as medical electrical leads including such components.

In Example 1, a medical electrical lead comprises a proximal end including a connector adapted to be operatively coupled to a pulse generator and a distal end adapted to be disposed at a location within a patient's body. The lead further comprises a lead body, a conductor, and at least one electrode. The lead body extends between the proximal end and the distal end and comprises a distal portion having at least one tubular insulating layer comprising a fiber reinforced silicone elastomer comprising a plurality of discrete, non-continuous fibers having a random orientation uniformly dispersed within a silicone elastomer matrix. The at least one conductor is operatively coupled to the connector and extends within the lead body from the proximal end to the distal end. The at least one electrode is located on the lead body and is operatively coupled to the at least one conductor.

In Example 2, the medical electrical lead according to Example 1, wherein the fiber reinforced silicone elastomer comprises about 30% to about 70% discrete fibers by volume.

In Example 3, the medical electrical lead according to either Example 1 or 2, wherein the fibers have an average length ranging from about 0.001 to about 0.070 inches.

In Example 4, the medical electrical lead according to any of Examples 1-3, wherein the average length of the fibers is less than a thickness of the tubular insulating layer.

In Example 5, the medical electrical lead according to any of Examples 1-4, wherein the fibers comprise a material selected from the group consisting of polyurethanes, polyesters, polyamides, polyacrylates, polyethylene terephthalate, polyaryletheretherketone, and polytetrafluoroethylene.

In Example 6, the medical electrical lead according to any of Examples 1-5, wherein the fibers comprise a thermoplastic polymer having a glass transition temperature greater than that of the silicone elastomer matrix processing temperature.

In Example 7, the medical electrical lead according to any of Examples 1-6, wherein the silicone elastomer matrix is any one of a 40 to 80 durometer silicone elastomer.

In Example 8, the medical electrical lead according to any of Examples 1-7, wherein the fibers comprise polyester fibers.

In Example 9, the medical electrical lead according to any of Examples 1-8, wherein the fibers comprise polyester monofilament fibers and the silicone elastomer matrix comprises a 40 to 80 durometer silicone elastomer.

In Example 10, the medical electrical lead according to any of Examples 1-9, wherein the fibers are pre-treated to promote adhesion between the fibers and the silicone elastomer matrix.

In Example 11, the medical electrical lead according to any of Examples 1-10, wherein the fiber reinforced silicone elastomer has an ultimate strain ranging from about 50 to about 150%.

According to Example 12, a medical electrical lead comprising a proximal end including a connector adapted to be operatively coupled to a pulse generator and a distal end adapted to be disposed at a location within a patient's body. The lead further comprises a lead body, at least one conductor, a first electrode and a second electrode. The lead body extends between the proximal end and the distal end. The at least one conductor is operatively coupled to the connector and extends within the lead body from the proximal end to the distal end. The first electrode and the second electrode are located on the lead body and are operatively coupled to the conductor. A portion of the lead body extending between the first and second electrodes comprises at least one tubular insulating layer comprising a fiber reinforced silicone elastomer including a plurality of discrete, non-continuous fibers having a random orientation uniformly dispersed within a silicone elastomer matrix.

In Example 13, the medical electrical lead according to Example 12, wherein the fiber reinforced silicone elastomer comprises about 30% to about 70% discrete fibers by volume.

In Example 14, the medical electrical lead according to Example 12 or 13, wherein the fibers have an average length ranging from about 0.001 to about 0.070 inches and wherein the average length of the fibers is less than a thickness of the tubular insulating layer.

In Example 15, the medical electrical lead according to any of Examples 12-14, wherein the fibers comprise a material selected from the group consisting of: polyurethanes, polyesters, polyamides, polyacrylates, polyethylene terephthalate, polyaryletheretherketone, and polytetrafluoroethylene.

In Example 16, the medical electrical lead according to any of Examples 12-15, wherein the fibers comprise a thermoplastic polymer having a melt temperature greater than that of the silicone elastomer matrix processing temperature.

In Example 17, the medical electrical lead according to any of Examples 12-16, wherein the fiber reinforced silicone elastomer has an ultimate strain ranging from about 50 to about 150%.

In Example 18, the medical electrical lead according to any of Examples 12-17, wherein the fibers comprise polyester fibers.

In Example 19, the medical electrical lead according to any of Examples 12-18, wherein the fibers comprise polyester monofilament fibers and the silicone elastomer matrix comprises a 40 to 80 durometer silicone elastomer.

In Example 20, a component for an implantable medical electrical lead body. The component comprises at least one layer including a fiber reinforced silicone elastomer, wherein the fiber reinforced silicone elastomer comprises about 30% to about 70% by volume of discrete fibers having a random orientation uniformly dispersed within a silicone elastomer matrix.

In Example 21, the component for an implantable medical electrical lead body according to Example 20, wherein the fiber reinforced silicone elastomer has an ultimate strain ranging from about 50 to about 150%.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a perspective view of a medical electrical lead according to an embodiment of the present invention.

FIG. 2 is a schematic view of a fiber reinforced silicone elastomer including a dispersion of discrete randomly oriented fibers in a silicone elastomer matrix utilized in the lead of FIG. 1 according to an embodiment of the present invention.

FIG. 3A is a schematic, longitudinal cross sectional view of a portion of a medical lead body according to one embodiment of the present invention.

FIG. 3B is an expanded view of area A from FIG. 3A according to an embodiment of the present invention.

FIG. 4 is a schematic longitudinal, cross sectional view of a portion of a medical lead body according to another embodiment of the present invention.

FIGS. 5A-5C are end cross-sectional views of a portion of a medical electrical lead body according to yet other embodiments of the present invention.

FIG. 6 is a longitudinal cross-section of a distal portion of a medical electrical lead body according to other embodiments of the present invention.

FIG. 7 is a longitudinal cross-sectional view of a portion of a lead body including a seal according to still other embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

The leads according to the various embodiments of the present invention are suitable for sensing intrinsic electrical activity and/or applying therapeutic electrical stimuli to a patient. Exemplary applications include, without limitation, cardiac rhythm management (CRM) systems and neurostimulation systems. For example, in exemplary CRM systems utilizing pacemakers, implantable cardiac defibrillators, and/or cardiac resynchronization therapy (CRT) devices, the medical electrical leads according to the various embodiments of the invention can be endocardial leads configured to be partially implanted within one or more chambers of the heart so as to sense electrical activity of the heart and apply a therapeutic electrical stimulus to the cardiac tissue within the heart. Additionally, the leads formed according to the various embodiments of the present invention may be suitable for placement in a coronary vein adjacent to the left side of the heart so as to facilitate bi-ventricular pacing in a CRT or CRT-D system. Still additionally, leads formed according to embodiments of the present invention may be configured to be secured to an exterior surface of the heart (i.e., as epicardial leads).

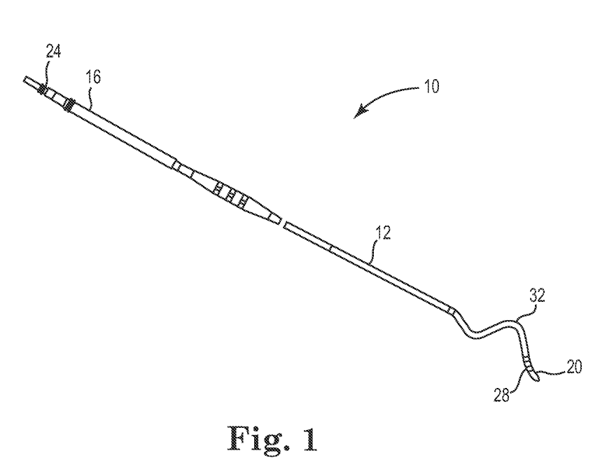

FIG. 1 is a perspective view of a medical electrical lead 10, according to various embodiments of the present invention. According to some embodiments, the medical electrical lead 10 can be configured for implantation within a patient's heart. According to other embodiments, the medical electrical lead 10 is configured to stimulate a nerve or bundle of nerves from an adjacent vessel. For example, the medical electrical lead 10 can be configured to stimulate the vagus nerve from a location within the internal jugular vein.

As shown in FIG. 1, the medical electrical lead 10 includes an elongated, insulative lead body 12 extending from a proximal end 16 to a distal end 20. The proximal end 16 is configured to be operatively connected to a pulse generator via a connector 24. The pulse generator is adapted to generate an electrical stimulus pulse used to stimulate the targeted region of the patient's body. At least one conductor extends from the connector 24 at the proximal end 16 of the lead 10 to one or more electrodes 28 at the distal end 20 of the lead 10. The conductor can be a coiled or cable conductor. According to some embodiments where multiple conductors are employed, the lead 10 can include a combination of coiled and cable conductors. When a coiled conductor is employed, according to some embodiments, the conductor can have either a co-radial or a co-axial configuration.

The lead body 12 is flexible and has a circular cross-section. According to one embodiment of the present invention, an outer diameter of the lead body 12 ranges from about 2 to about 15 French. The medical electrical lead 10 may be unipolar, bipolar, or multi-polar depending upon the type of therapy to be delivered and/or the operational requirements of the cardiac rhythm management or neurostimulation system. In embodiments of the present invention employing multiple electrodes 28 and multiple conductors, each conductor can be adapted to be connected to an individual electrode 28 in a one-to-one manner allowing each electrode 28 to be individually addressable. In other embodiments employing multiple electrodes, one or more electrodes 28 can be adapted to be addressable via a single conductor path.

The electrodes 28 can have any electrode configuration as is known in the art. According to one embodiment of the present invention, at least one electrode can be a ring or partial ring electrode. According to another embodiment, at least one electrode is a shocking coil. According to yet another embodiment of the present invention, at least one electrode 28 includes an exposed electrode portion and an insulated electrode portion. In some embodiments, a combination of electrode configurations may be used. The electrodes 28 can be coated with or formed from platinum, stainless steel, MP35N, a platinum-iridium alloy, or another similar conductive material. In further embodiments, a steroid eluting collar may be located adjacent to at least one electrode 28.

According to various embodiments, the lead body 12 can include one or more fixation features for securing and stabilizing the lead body 12 including the one or more electrodes 28 at a target site within a patient's body. The fixation feature(s) can be passive or active. Examples of passive fixation features include a pre-formed distal portion 32 (FIG. 1) of the lead body 12 adapted to bear against the vessel walls and/or expandable tines provided at the distal end of the lead body 12. An exemplary active fixation member includes a screw-in fixation member. In some embodiments, the fixation member can be an extendable/retractable fixation member and can include one or more mechanical components adapted to facilitate the extension/retraction of the fixation member. An exemplary extendable/retractable fixation member is shown and described in U.S. Pat. No. 6,463,334 which is herein incorporated by reference.

In various embodiments, the lead body 12 is made from one or more biocompatible, electrically insulative materials selected such that the lead will exhibit desired physical and operational characteristics. In one embodiment, at least one major portion of the lead body 12 is manufactured from a relatively stiff polymeric material, e.g., polyurethane, while another portion, e.g., a distal portion or segment, is made from a relatively soft and flexible material such as a silicone rubber or a copolymer thereof. Configuring the distal portion of the lead body 12 to be relatively soft and flexible advantageously facilitates navigation of the tortuous pathways of a patient's vasculature system to reach the target stimulation site, while configuring the more proximal portion to be relatively stiff enhances pushability and torque transfer along the lead body 12 for delivery. In various embodiments, as explained in further detail below, the silicone rubber polymeric materials used in the various portions of the lead body are reinforced to increase the materials' resistance to stretching under tension, while at the same time maintaining its flexibility and increasing its tear resistance.

According to various embodiments, the material used to construct at least a portion of the lead body 12 includes a fiber-reinforced silicone elastomer. In one embodiment, the fiber reinforced silicone elastomer includes a dispersion of discrete, non-continuous fibers in a silicone elastomer matrix. A fiber reinforced silicone elastomer will exhibit improved mechanical properties such as, for example, stiffness and strength, relative to non-reinforced silicone elastomers. Additionally, the overall mechanical properties of the fiber-reinforced silicone elastomer can be manipulated to achieve a desired set of mechanical properties by varying the amount of fiber used to reinforce the silicone elastomer, the type of fiber, the length of the fiber, and/or the orientation of the fibers dispersed within the silicone elastomer. Further, as the fiber-reinforcement of the silicone elastomer enhances the mechanical properties of the silicone elastomer, reduced wall thicknesses can be achieved without a decrease in the overall mechanical strength and stiffness of the material. In one embodiment, the fiber reinforced silicone elastomer has an ultimate strain ranging from about 50 to about 150%.

The silicone elastomer used to form the fiber reinforced silicone elastomer can be any silicone elastomer, whether now known or later developed, that is suitable for use in biomedical applications. Some exemplary silicone elastomers suitable for use in the present invention include the NuSil family of silicones available from NuSil Technology LLC of Carpinteria, Calif. In one embodiment, the silicone elastomer employed in the matrix is any one of a 40 to 80 durometer silicone elastomer. In another embodiment, the silicone elastomer is a 60 durometer silicone elastomer.

The reinforcing fibers dispersed within and used to reinforce the silicone elastomer can be natural or synthetic fibers. In some embodiments, the reinforcing fibers can include a combination of natural and synthetic fibers. In other embodiments, the fibers can be thermoplastic or thermoset fibers. Exemplary reinforcing fibers include, but are not limited to: polyurethane fibers, polyester fibers, polyamide fibers, polyisobutylene based polyurethane fibers, polyacrylate fibers, polyethylene terephthalate (e.g., Dacron®) fibers, polyaryletheretherketone (PEEK) fibers, and polytetrafluoroethylene (PTFE) fibers, among others. In one embodiment, the reinforcing fibers are selected such that the glass transition temperature T_{g} of the reinforcing fibers is higher than the processing temperature of the silicone elastomer matrix throughout which they are dispersed. According to some embodiments, the reinforcing fibers are processed as a monofilament using standard extrusion methods. The monofilament can then be braided or twisted into the fiber.

The reinforcing fibers can be long or short fibers. In one embodiment, the reinforcing fibers are short, discrete fibers having a length ranging from about 0.001 inches to about 0.070 inches. In one embodiment, the fiber length is selected such that it is less than the average wall thickness of the portion of the lead body 12 or lead body component constructed from the fiber-reinforced silicone elastomer. In other embodiments, the reinforcing fibers incorporated into the silicone elastomer can include a mix of both long and short fibers.

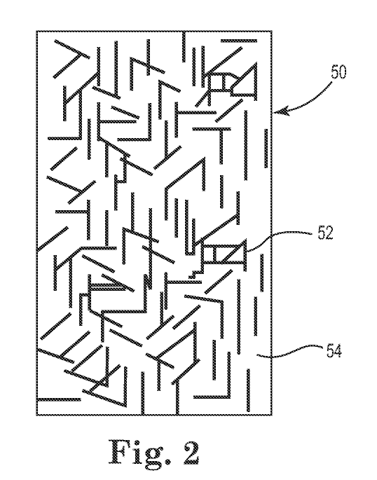

FIG. 2 is a schematic view of a fiber reinforced silicone elastomer matrix 50 suitable for use in the lead body 12, showing discrete fibers 52 dispersed within a silicone elastomer matrix 54. The reinforcing fibers are dispersed within the silicone elastomer matrix prior to being molded or extruded into the desired portion of the lead body 12. In one embodiment, the fiber reinforced silicone elastomer is extruded and then assembled with various conductive components to form the lead body 12. The amount of fibers dispersed within the silicone elastomer matrix can range from about 30% to about 70% by total volume of the elastomeric matrix. In one embodiment, the reinforcing fibers are uniformly dispersed throughout the silicone elastomer matrix. When dispersed within the silicone elastomer matrix, the reinforcing fibers can have either a two-dimensional orientation (oriented along either the X-axis or Y-axis) or a random, three-dimensional orientation. In one embodiment, as shown in FIG. 2, the reinforcing fibers are short, discrete fibers having a random orientation and are uniformly dispersed within the silicone elastomer. The use of randomly oriented fibers within the silicone elastomer matrix permits the material to be molded or extruded without having to control fiber position and orientation. This minimizes the number or processing steps used during the manufacturing process.

In certain embodiments, depending upon the properties of the matrix material, the fibers may undergo a pre-treatment step prior to being dispersed within the silicone elastomer matrix. The pre-treatment step may facilitate better adhesion between the fiber and the matrix material. In one embodiment, for example, the fibers may be pre-treated using plasma, primer, or other surface treatment techniques. In another embodiment, the fibers may be impregnated with a silicone prior to being dispersed within the silicone elastomer matrix. In some embodiments, the pre-treatment of fibers prior to their incorporation into the matrix material is not necessary.

In one embodiment, the fiber reinforced silicone elastomer includes a plurality of discrete, randomly oriented polyester monofilament fibers uniformly dispersed throughout a silicone rubber matrix. In one embodiment, the polyester monofilament fibers are Mersilene fibers available from Ethicon, Inc. and the silicone rubber matrix is NuSil 60 durometer LSR available from NuSil Technology LLC of Carpinteria, Calif. In a further embodiment, the polyester monofilament fibers have an average diameter of about 0.0005 inches and an average length of about 0.002 inches.

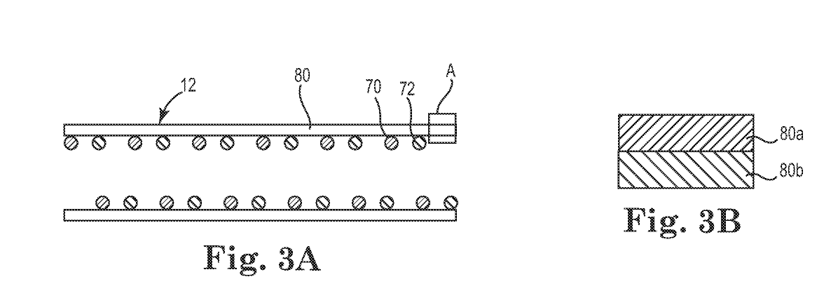

In some embodiments, the fiber reinforced silicone elastomer can be extruded or molded into a portion or portions of the lead body 12. In one embodiment, the entire lead body 12 is constructed from a fiber-reinforced silicone elastomer. Various lead body configurations that can be constructed using the fiber reinforced silicone elastomers, described above according to the various embodiments, will now be discussed in more detail in reference to FIGS. 3A-5C. FIG. 3A is a schematic longitudinal cross sectional view of an insulated (non-electrode) portion of a lead body 12 provided in accordance with various embodiments of the present invention. According to one embodiment, as shown in FIG. 3A, the lead body 12 includes a first coiled conductor 70 and a second coiled conductor 72 disposed in a co-radial arrangement with one another. The coiled conductors 70, 72 can be made, for example, of stainless steel, Eigiloy, or MP35N, among other suitable conductive materials. In some embodiments, the coiled conductors 70, 72 may each be provided with an individual layer of insulating material, for example, a low-friction polymeric material such as polytetrafluoroethylene (PTFE) or ethylene-tetrafluoroethylene fluoropolymer (ETFE), among other low-friction fluoropolymers and non-fluoropolymers.

As shown in FIG. 3A, the coiled conductors 70, 72 are disposed within at least one tubular insulation layer 80, which acts to chemically, mechanically and electrically insulate the coiled conductors 70, 72 from the surrounding external environment, and can also provide the lead body 12 with the desired mechanical properties. According to one embodiment, the tubular insulation layer 80 can be fabricated from a fiber reinforced silicone elastomer as discussed above according to the various embodiments.

In another embodiment, the tubular insulation layer 80 can include two or more layers of polymeric material, which can form two or more coaxial tubular material regions as shown in FIG. 3B. FIG. 3B is an expanded view of area “A” of FIG. 3A. As shown in FIG. 3B, the tubular insulation layer 80 includes two coaxial tubular material regions 80a and 80b. The two coaxial tubular material regions 80a and 80b can be formed from the same or different materials. For example, in one embodiment, the outer material region 80a can be formed from a fiber reinforced silicone elastomer such as described above according to the various embodiments and the inner material region 80b can be formed from a non-reinforced silicone rubber or polyurethane. In another embodiment, both the inner and outer tubular material regions 80a and 80b can be formed from a fiber reinforced silicone elastomer such as described above according to the various embodiments.

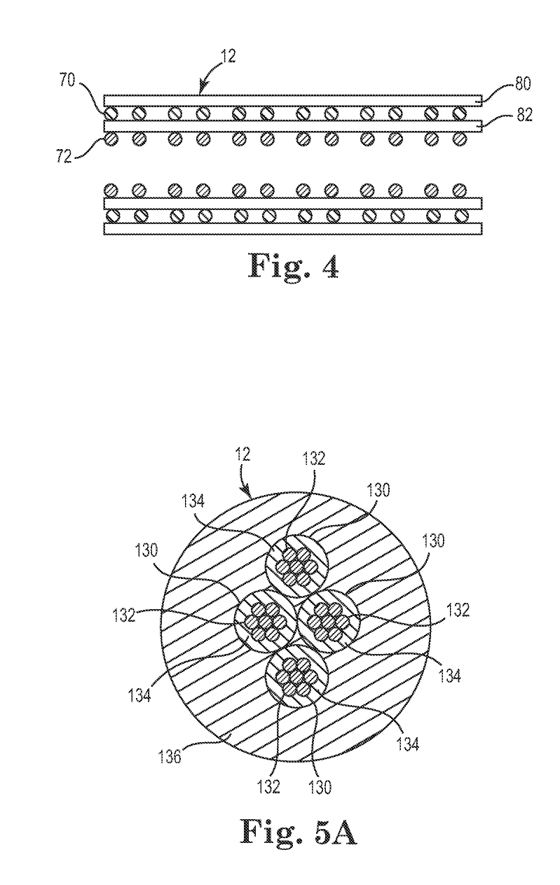

Like FIG. 3A, FIG. 4 is a schematic longitudinal cross sectional view of an insulated portion of lead body 12 according to another embodiment of the invention. As shown in FIG. 4, the lead body 12 includes first and second coiled conductors 70, 72, each of which may be provided with a layer of a suitable insulating material, for example, a fluoropolymer such as those described above, among others. As shown in FIG. 4, the first and second coiled conductors 70, 72 are disposed in a co-axial (rather than co-radial) arrangement with one another. The inner coiled conductor 72 is provided with a tubular insulation layer 82, which acts to insulate the coiled conductor 72 from the external environment and also from the outer coiled conductor 70. The inner tubular insulation layer 82 may also provide the lead body 12 with the desired mechanical characteristics. In one embodiment, the inner tubular insulation layer 82 is formed from a fiber reinforced silicone elastomer such as described above according to the various embodiments of the present invention.

As shown in FIG. 4, the outer coiled conductor 70 is disposed over the inner tubular insulation layer 82, and an outer tubular insulation layer 80 is disposed over the outer coiled conductor 70. Like the inner tubular insulation layer 82, the outer tubular insulation layer 80 of FIG. 4 also can be formed from a fiber reinforced silicone elastomer such as described above according to the various embodiments. The outer tubular insulation layer 80 of FIG. 4 can also comprise two or more material regions, for example, two or more layers of material, which may form two or more coaxial tubular material regions. An exemplary two-material region suitable for use in the outer tubular insulation layer 80 of the lead body 12 shown in FIG. 4 is discussed above in reference to FIG. 3B.

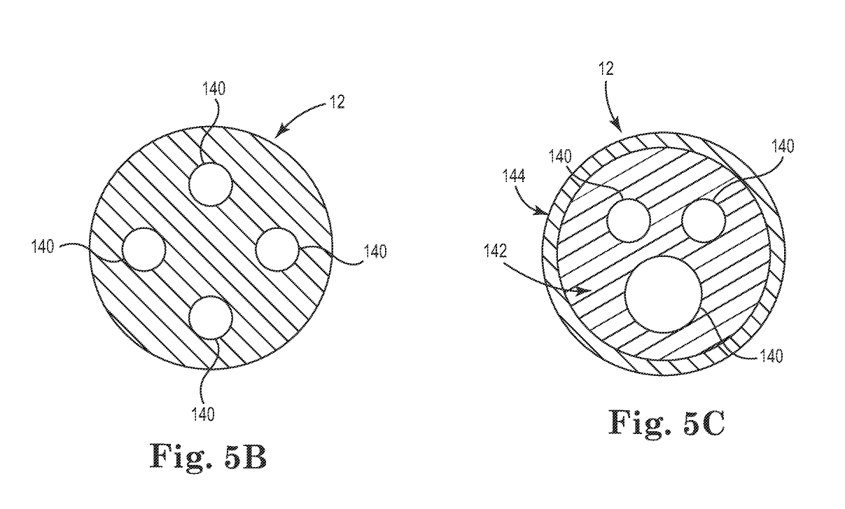

FIG. 5A is an end cross-sectional view of the lead body 12 according to yet another embodiment of the present invention. As shown in FIG. 5A, the insulative lead body 12 includes a plurality of cable conductors 130, each having a plurality of conductive filaments 132. The filaments 132 may be separately insulated from one another. According to one embodiment the cable conductors 130 can include at least one layer of insulation 134 provided over their outer periphery. In one embodiment, the outer tubular insulation 136 forming the lead body 12 can be co-extruded along with the cable conductors 130 or the individual filaments 132 forming the cable conductors 130. In another embodiment, the outer insulation 136 can be molded around the cable conductors 130. In one embodiment, the outer tubular insulation 136 can be formed from a fiber reinforced silicone elastomer such as described above according to the various embodiments.

FIGS. 5B and 5C are cross-sectional views of a lead body 12 according to still other embodiments of the present invention. As shown in FIG. 5B, the lead body 12 is formed such that it includes four lumens 140, although any number of lumens can be provided. In one embodiment, the lead body 12 is formed from a fiber reinforced silicone elastomer such as described above according to the various embodiments and can be either extruded or molded. As shown in FIG. 5C, the lead body 12 includes an inner core member 142 including multiple lumens 140 and at least one outer tubular insulation layer 144. In some embodiments, both the inner core member 142 and the outer tubular insulation layer 144 can be formed from a fiber reinforced silicone elastomer such as described above. In another embodiment, only the outer tubular insulation layer 144 is formed from a fiber reinforced silicone elastomer.

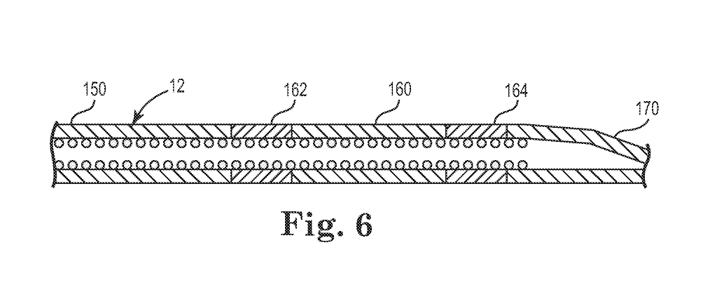

In some embodiments, a fiber reinforced silicone elastomer according to the various embodiments described above is used to form a select portion of the lead body 12. FIG. 6 is a longitudinal cross-section of a distal portion 150 of a lead body 12. In one embodiment, a fiber reinforced silicone elastomer is used to fabricate the distal portion 150 of a lead body 12. In another embodiment, a fiber reinforced silicone elastomer can be used to fabricate a portion 160 of the lead body 12 extending between a proximal electrode 162 and a distal electrode 164. In still other embodiments, a fiber reinforced silicone elastomer is used to fabricate the distal tip 170 of the lead body. The use of a fiber reinforced silicone elastomer in each of these regions may also allow a reduction in wall thickness and an overall reduction in the outer diameter of the lead body without a decrease in the overall mechanical strength and stiffness of the material.

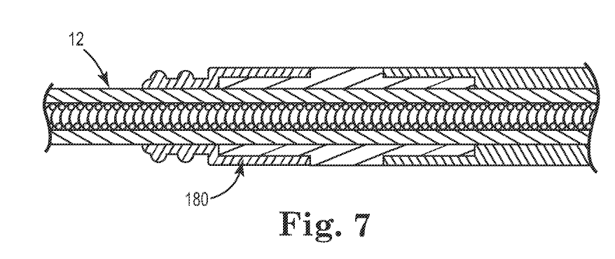

Finally, according to still other embodiments of the present invention, various lead body components can be formed from a fiber reinforced silicone elastomer described above according to the various embodiments. A lead body component including a fiber reinforced silicone elastomer may exhibit improved dimensional control during processing and improved creep resistance relative to components made of non-reinforced silicone materials. The various lead body components formed from a fiber reinforced silicone elastomer provided in accordance with various embodiments of the present invention can either be extruded or molded. In another embodiment, a fiber reinforced silicone elastomer can be molded over an existing lead body component. Such lead components include, but are not limited to: lead tips, O-rings, seals, suture sleeves and other components useful in lead body construction. FIG. 7 is a longitudinal cross-sectional view of a portion of a lead body 12 including a seal 180. In one embodiment, the seal 180 is fabricated from a fiber reinforced silicone elastomer such as described above according to the various embodiments.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

I claim:

1. A medical electrical lead comprising:
a proximal end including a connector adapted to be operatively coupled to a pulse generator and a distal end adapted to be disposed at a location within a patient's body;
a lead body extending between the proximal end and the distal end, the lead body comprising a distal portion having at least one tubular insulating layer comprising a fiber reinforced silicone elastomer comprising a plurality of discrete, non-continuous fibers having a random orientation uniformly dispersed within a silicone elastomer matrix;
at least one conductor operatively coupled to the connector and extending within the lead body from the proximal end to the distal end; and
at least one electrode located on the lead body and operatively coupled to the at least one conductor.

2. The medical electrical lead according to claim 1, wherein the fiber reinforced silicone elastomer comprises about 30% to about 70% discrete fibers by volume.

3. The medical electrical lead according to claim 1, wherein the average length of the fibers is less than a thickness of the tubular insulating layer.

4. The medical electrical lead according to claim 1, wherein the fibers comprise a material selected from the group consisting of polyurethanes, polyesters, polyamides, polyacrylates, polyethylene terephthalate, polyaryletheretherketone, and polytetrafluoroethylene.

5. The medical electrical lead according to claim 1, wherein the fibers comprise a thermoplastic polymer having a glass transition temperature greater than that of the silicone elastomer matrix processing temperature.

6. The medical electrical lead according to claim 1, wherein the silicone elastomer is any one of a 40 to 80 durometer silicone elastomer.

7. The medical electrical lead according to claim 1, wherein the fibers comprise polyester fibers.

8. The medical electrical lead according to claim 1, wherein the fibers comprise polyester monofilament fibers and the silicone elastomer matrix comprises a 40 to 80 durometer silicone elastomer.

9. The medical electrical lead according to claim 1, wherein the fibers are pre-treated to promote adhesion between the fibers and the silicone elastomer matrix.

10. The medical electrical lead according to claim 1, wherein the fiber reinforced silicone elastomer has an ultimate strain ranging from about 50 to about 150%.

11. A medical electrical lead comprising:
a proximal end including a connector adapted to be operatively coupled to a pulse generator and a distal end adapted to be disposed at a location within a patient's body:
a lead body extending between the proximal end and the distal end;
a plurality of conductors operatively coupled to the connector and extending within the lead body from the proximal end to the distal end; and
a first electrode and a second electrode located on the lead body, each operatively coupled to one of the plurality of conductors, wherein a portion of the lead body extending between the first and second electrodes comprises at least one tubular insulating layer comprising a fiber reinforced silicone elastomer including a plurality of discrete, non-continuous fibers having a random orientation uniformly dispersed within a silicone elastomer matrix.

12. The medical electrical lead according to claim 11, wherein the fiber reinforced silicone elastomer comprises about 30% to about 70% discrete fibers by volume.

13. The medical electrical lead according to claim 11, wherein the average length of the fibers is less than a thickness of the tubular insulating layer.

14. The medical electrical lead according to claim 11, wherein the fibers comprise a material selected from the group consisting of: polyurethanes, polyesters, polyamides, polyacrylates, polyethylene terephthalate, polyaryletheretherketone, and polytetrafluoroethylene.

15. The medical electrical lead according to claim 11, wherein the fibers comprise a thermoplastic polymer having a melt temperature greater than that of the silicone elastomer matrix processing temperature.

16. The medical electrical lead according to claim 11, wherein the fiber reinforced silicone elastomer has an ultimate strain ranging from about 50 to about 150%.

17. The medical electrical lead according to claim 11, wherein the fibers comprise polyester fibers.

18. The medical electrical lead according to claim 11, wherein the fibers comprise polyester monofilament fibers and the silicone elastomer matrix comprises a 40 to 80 durometer silicone elastomer.

19. A component for an implantable medical electrical lead body, the component comprising at least one layer including a fiber reinforced silicone elastomer, wherein the fiber reinforced silicone elastomer comprises about 30% to about 70% by volume of discrete fibers having a random orientation uniformly dispersed within a silicone elastomer matrix.

20. The component according to claim 19, wherein the fiber reinforced silicone elastomer has an ultimate strain ranging from about 50 to about 150%.
